(19) United States
(12) Patent Application Publication
(10) Pub. No.: US 2008/0163706 A1
(43) Pub. Date: Jul. 10, 2008

(54) Water leak disclosing agent and water leak detection method

(76) Inventor: Wen-Kuo Hsu, Taoyuan (TW)

Correspondence Address: TROXELL LAW OFFICE PLLC, Suite 1404, 5205 Leesburg Pike, Falls Church, VA, 22041, US

(21) Appl. No.: 11/651,613

(22) Filed: Jan. 10, 2007

(51) Int. Cl. G01N 33/18; G01M 3/08

(52) U.S. Cl. 73/865.8; 252/408.1

(57) ABSTRACT

A water leak disclosing agent and a water leak detection method are disclosed. The water leak disclosing agent is a pigment-containing colored tablet or a pigment solution, wherein the pigment material stains the water if it is put thereinto so it is adopted as the disclosing agent for water leak detection. In accordance with the water leak detection method, the colored tablet is plunged into a water pipe or a floorslab of a floor or a bathroom is filled with the pigment solution. As a result, the leaky position can be identified easily and correctly according to the color of the oozed colored liquid.

### FIELD OF THE INVENTION

The present invention relates to a water leak disclosing agent and a water leak detection method, wherein the colored disclosing tablets are applied to the leaky location so that the leaky pipe or the leaky position can be identified according to the color of the oozed colored liquid.

### BACKGROUND OF THE INVENTION

Once the water pipes, which are generally hidden in the floorslabs, floors and walls, are leaky, the leaky positions must be found out and repaired. In accordance with the method of detecting the leaky position, the inlet of the water tower, the motor of the water tower, the inlet of the stool, and all hydrants are closed, individually. Next, the reading on the water gauge and the position of the small triangular rotation shaft of the water gauge are recorded. After few hours or a night of waiting, the closing time and the check time are recorded. Next, the leaky positions can be identified by making a one-by-one check. However, this kind of detection method takes much more time and only the rough range can be identified.

In addition, in accordance with another detection method, a fluorescent agent acting as a detection agent is added into the water pipe so that when the fluorescent agent is oozed from the water leak position, the water leak position can be identified by irradiation of ultraviolet rays. However, the fluorescent agent is diluted easily after mixing with water. As a result, the addition amount of the fluorescent agent must be increased so as to expose the water leak position normally. However, the fluorescent agent contains toxic materials. In addition, the fluorescent agent adheres to the pipe walls easily so it is not easy to discharge the fluorescent agent from the water within a short period of time. The fluorescent agent-containing water is harmful and accumulated in the human body once the user touches it.

Up to now, there is disclosed no method to solve the water leak problem quickly and effectively.

### SUMMARY OF THE INVENTION

In view of the above-mentioned conventional defects, a major object of the present invention is to provide a water leak disclosing agent and a water leak detection method, which reduce cost and provide superior performance.

The disclosing agent of the present invention is a pigment-containing colored tablet into which the edible pigment material is doped, wherein the edible pigment material comprises the following distinguishable colors: Blue Nos. 1 and 2; Green No. 2; Yellow Nos. 4 and 5; and Red Nos. 6, 7 and 40. The edible pigment material is soluble in water. Accordingly, the edible pigment material stains the water if it is put thereinto so it is adopted as the disclosing agent for water leak detection. The pigment materials described above are statutory artificial edible. As a result, both the colored tablet, which is plunged into the water pipeline system, and the pigment solution are harmless to the human body.

In addition, the colored tablet can be covered with a layer of water-soluble coating so as to prevent the hands from contamination by the colored tablet. The water-soluble coating is such as a sugar coating or other edible and water-soluble material.

The pigment solution is suitable for use in a large-area bathroom or floorslab if it is doped with industrial pigment material.

In accordance with the detection method of the present invention, the water pipeline system is closed beforehand, and the colored tablets having respective different color pigment materials are then plunged into the respective water pipes of the water pipeline system to be detected. Next, the water pipeline system is opened again so as to dissolve the colored tablets. Next, a check is made on the outer walls of the water pipes to determine whether a colored liquid is oozed out. As a result, the leaky pipe can be identified immediately according to the color of the oozed colored liquid.

In accordance with the detection method of the present invention, the floorslab of the floor or the bathroom, which is suspected to be leaky, is filled with the industrial pigment solution. Next, a check is made on the underneath floor to determine whether the colored pigment liquid is oozed out. As a result, the leaky position can be identified easily and correctly according to the color of the oozed colored liquid.

### BRIEF DESCRIPTION OF THE DRAWINGS

FIG. 1 is a schematic diagram showing the colored tablets are plunged into the water pipes in accordance with the present invention.

FIG. 2 is a schematic enlarged view of the encircled region in FIG. 1.

FIG. 3 is a schematic diagram showing that the floorslab is filled with the pigment solution in accordance with the present invention.

### DETAILED DESCRIPTION OF THE PREFERRED EMBODIMENT

The present invention relates to a water leak disclosing agent and a water leak detection method, wherein the water leak disclosing agent is generally a pigment-containing colored tablet or a pigment solution, wherein the pigment material stains the water if it is put thereinto so it is adopted as the disclosing agent for water leak detection.

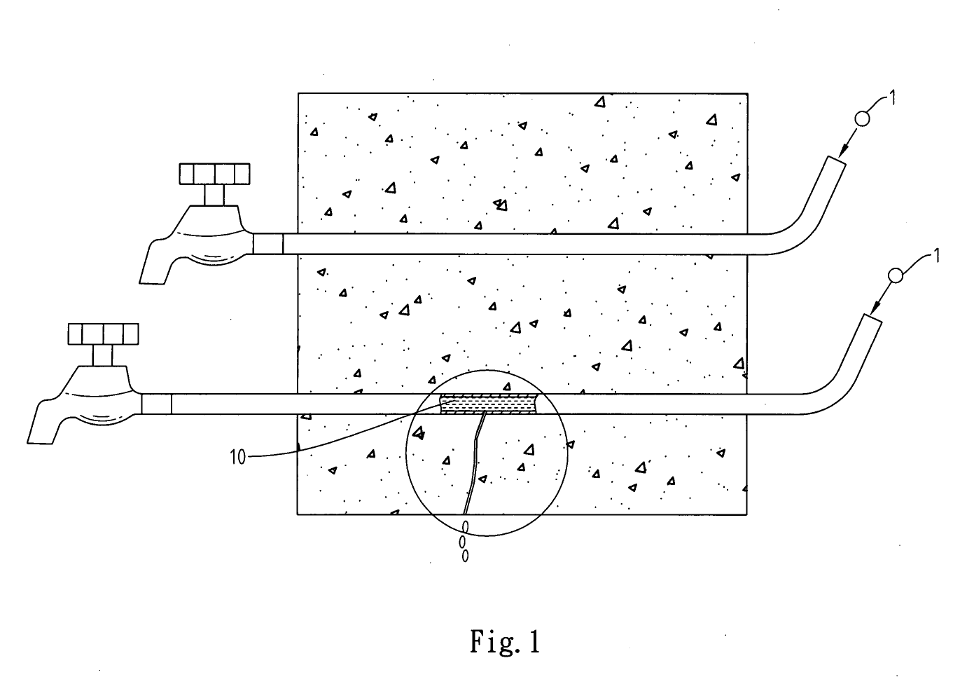

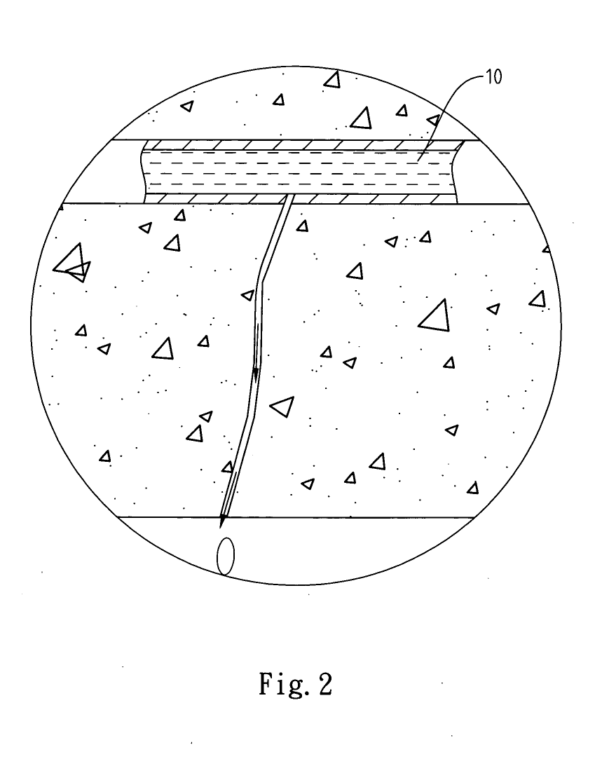

Referring to FIG. 1 and FIG. 2, the water leak disclosing agent and the water leak detection method are shown. As shown in these figures, the disclosing agent is applied to a water pipeline system, wherein the disclosing agent is a kind of colored tablet 1.

The disclosing agent is formed by fully mixing a binder (20%) with a powder of edible pigment material (80%) whose mixture is then compressed into the colored tablet 1. The edible pigment material comprises the following distinguishable colors: Blue Nos. 1 and 2; Green No. 2; Yellow Nos. 4 and 5; and Red Nos. 6, 7 and 40. The edible pigment material is soluble in water. Accordingly, the edible pigment material stains the water if it is put thereinto so it is adopted as the disclosing agent for water leak detection.

The binder comprises water and adhesive flour, wherein the adhesive flour is composed of an edible material such as cornmeal, lactose, potato starch, or the like. Various colored tables 1 of various colors can be formed by doping various edible pigment materials thereinto.

The colored tablet 1 is applicable to the water pipeline system, which is a general water intake pipe or a hot water pipe. In accordance with the water leak detection method of the present invention, the water pipeline system is closed beforehand, and the colored tablets 1 having respective different color pigment materials are then plunged into the respective water pipes to be detected. Next, the water pipeline system is opened again so as to dissolve the colored tablets 1. Next, a check is made on the outer walls of the water pipes to determine whether a colored liquid 10 is oozed out. As a result, the leaky pipe of these water pipes can be identified immediately according to the color of the oozed colored liquid 10.

Moreover, the colored tablets 1 are covered with a layer of water-soluble coating so as to prevent the hands from contamination by the colored tablets 1. The water-soluble coating is such as a sugar coating or other edible and water-soluble material.

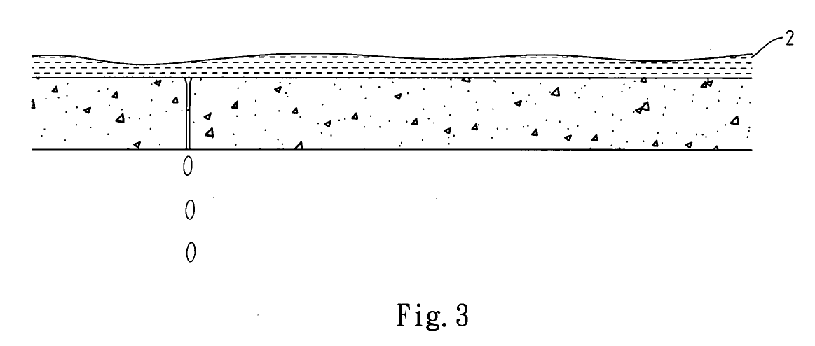

Referring further to FIG. 3, the disclosing agent is applied to a floorslab of a floor or a bathroom, wherein the disclosing agent is a kind of pigment solution 2.

The disclosing agent is formed by fully mixing a water solution (70%) with a powder of industrial pigment material (30%) so as to form the pigment solution 2. Various pigment solutions 2 of various colors can be formed by doping various industrial pigment materials thereinto.

In accordance with the detection method, the floorslab of the floor or the bathroom, which is suspected to be leaky, is filled with the pigment solution 2. Next, a check is made on the underneath floor to determine whether the colored pigment liquid is oozed out. Accordingly, the leaky position can be identified easily and correctly.

The water leak disclosing agent and the water leak detection method of the present invention are creative and novel and achieve the expected purposes and effects. In comparison with the conventional detection method, the creative designs of the present invention provide greater advantages.

What the invention claimed is:

1. A water leak disclosing agent and a water leak detection method comprising:
said water leak disclosing agent being a colored tablet formed by fully mixing a binder (20%) with a powder of edible pigment material (80%) whose mixture is then compressed; and
said water leak detection method comprising:
closing a water pipeline system;
plunging said colored tablet into a water pipe of said water pipeline system;
opening said water pipeline system so as to dissolve said colored tablet; and
checking whether a colored liquid is oozed to an outer wall of said water pipe.

2. A water leak disclosing agent and a water leak detection method of claim 1, wherein said binder comprises water and adhesive flour.

3. A water leak disclosing agent and a water leak detection method of claim 2, wherein said adhesive flour is cornmeal, lactose or potato starch.

4. A water leak disclosing agent and a water leak detection method of claim 1, wherein said colored tablet is covered with a layer of water-soluble coating.

5. A water leak disclosing agent and a water leak detection method of claim 1, wherein said colored tablet is covered with a layer of water-soluble coating, and said water-soluble coating is a sugar coating or other edible and water-soluble material.

6. A water leak disclosing agent and a water leak detection method comprising:
said water leak disclosing agent being formed by mixing a water solution (70%) with a powder of industrial pigment material (30%) fully so as to form a pigment solution; and
said water leak detection method comprising:
filling a floorslab of a floor or a bathroom with said pigment solution; and
checking an underneath floor to determine whether said pigment solution is oozed out.
